(19) United States
(12) Patent Application Publication
(10) Pub. No.: US 2006/0264362 A1
(43) Pub. Date: Nov. 23, 2006

(54) Method of modulating smooth muscle cell functioning by modulating sphingosine kinase mediated signalling

(75) Inventors: Stuart M. Pitson, Norwood (AU); Steffen-Sebastian Bolz, Munich (DE)

Correspondence Address: SEED INTELLECTUAL PROPERTY LAW GROUP PLLC, 701 FIFTH AVE, SUITE 6300, SEATTLE, WA, 98104-7092, US

(73) Assignee: Medvet Science Pty. Ltd., Stepney (AT)

(21) Appl. No.: 10/549,548

(22) PCT Filed: Mar. 18, 2004
(86) PCT No.: PCT/AU04/00336

§ 371 Date: Jun. 26, 2006

(30) Foreign Application Priority Data
Mar. 18, 2003 (AU) 2003901270

(51) Int. Cl. A61K 38/17

(52) U.S. Cl. 514/12

(57) ABSTRACT

The present invention relates generally to a method of modulating smooth muscle cell functioning and agents useful for same. More particularly, the present invention relates to a method of modulating smooth muscle tone by modulating intracellular sphingosine kinase mediated signalling. The method of the present invention is useful, inter alia, in the treatment and/or prophylaxis of conditions characterised by aberrant, unwanted or otherwise inappropriate smooth muscle tone, in particular aberrant, unwanted or otherwise inappropriate vascular, bronchial or intestinal smooth muscle tone.

### FIELD OF THE INVENTION

The present invention relates generally to a method of modulating smooth muscle cell functioning and agents useful for same. More particularly, the present invention relates to a method of modulating smooth muscle tone by modulating intracellular sphingosine kinase mediated signalling. The method of the present invention is useful, inter alia, in the treatment and/or prophylaxis of conditions characterised by aberrant, unwanted or otherwise inappropriate smooth muscle tone, in particular aberrant, unwanted or otherwise inappropriate vascular, bronchial or intestinal smooth muscle tone.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Early last century (1919), Bayliss described the intrinsic property of smooth muscle cells to react to stretch with an increase in tone. This effect is the basis for autoregulatory properties of resistance vessels that respond with a vasoconstriction to increases in transmural pressure (pressure-induced myogenic vasoconstriction), thereby providing “as far as possible for the maintenance of a constant flow of blood through the tissues supplied by them, whatever may be the height of the general blood pressure” (Bayliss, W. M. (1919) J Physiol Lond 28:220-231). Furthermore, the myogenic response directly affects systemic blood pressure and contributes up to two thirds of the increase in total peripheral resistance (Metting, P. J., Stein, P. M., Stoos, B. A. et al. (1989) Am J Physiol 256:R98-R105).

Blood pressure is the force exerted against artery walls as blood is carried through the circulatory system. The measurement of force is made in relation to the heart's pumping activity, and is measured in millimetres of mercury (mmHg). The systolic pressure is the measurement of pressure that occurs when the heart contracts or beats while diastolic pressure is the measurement recorded between beats, while the heart is relaxed.

Hypertension indicates that the force required for blood flow is greater than normal. According to the Sixth Report of the Joint National Committee on Detection, Evaluation and Treatment of High Blood Pressure (JNC VI), a blood pressure measurement of less than 130/85 is considered normal and 130 to 140/85 to 90 is defined as high-normal. Hypertension is generally classified as being primary or secondary. Primary hypertension has no known cause. However genetic and lifestyle factors such as body weight and salt consumption can contribute to high blood pressure. Eighty to ninety percent of persons diagnosed with hypertension fit in this category. This diagnosis is often made when no other cause can be found. Secondary hypertension is usually caused by the existence of another medical condition such as kidney disease, Cushing's syndrome, pregnancy, or chronic alcohol abuse. Oral contraceptives, prednisone, cyclosporin, and several other medications may also cause hypertension as a drug-related side effect.

There are several factors which put people at risk for hypertension. Increasing age, gender, heredity and race are factors that cannot be controlled. Elderly individuals are especially encouraged to undergo regular screening for the presence of hypertension because the condition is so prevalent in this population and is treatable once identified. Men are generally at greater risk than women. However, as women age, their risk increases with the onset of menopause such that later in life their risk exceeds men's. Heredity can be a risk factor if one or more parents are diagnosed with hypertension.

Controllable risk factors are lifestyle related: obesity, diet, lack of exercise, stress, the use of certain medications, smoking and excessive alcohol consumption.

For many people hypertension often remains undiagnosed when they are asymptomatic. However, some people may experience symptoms such as headache, dizziness, irregular or rapid heartbeat, nosebleeds, fatigue and blurred vision. It is estimated that one in four adults exhibits elevated blood pressure and more than 30 percent of them are unaware of this fact. Since people with hypertension may not exhibit any symptoms, their high blood pressure is often undiagnosed until complications occur.

The means by which the myogenic response in resistance arteries is initiated and developed remains largely unknown, but for limited information in this regard, in particular in relation to the coordinate regulation of the mechanisms which control the transmembrane influx of extracellular calcium Ca²⁺ versus the pressure dependent increase in myofilament calcium sensitivity to induce a highly reproducible reaction of arterial smooth muscle cells to pressure. Accordingly, in light of the significant health problem posed by the development of hypertension, there is an urgent need to elucidate the mechanisms which regulate vasoconstriction such that means of therapeutically and/or prophylactically treating inappropriate vascular smooth muscle tone can be developed.

In work leading up to the present invention, it has been surprisingly determined that resting tone and myogenic responses in resistance arteries are modulated by altering the expression and activity of sphingosine kinase. In particular, sphingosine kinase has been identified as the major determinant of microvascular tone and a leading candidate to orchestrate the two main components of the myogenic response. Without limiting the present invention to any one theory or mode of action, sphingosine kinase is an integral component of a pathway which translates mechanical force into intracellular signals and is therefore of significant importance to all cell types which translate mechanic stimuli into specific intracellular signals. The elucidation of this cellular signalling mechanism now facilitates the rational design of methodology directed to modulating smooth muscle constriction, in particular vascular, bronchial and intestinal smooth muscle constriction, by regulating the functioning of sphingosine kinase.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention is directed to a method of modulating smooth muscle cell activity, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

In another aspect there is more particularly provided a method of modulating vascular smooth muscle cell activity, said method comprising modulating the functional activity of sphingosine kinase mediated signalling wherein upregulating sphingosine kinase mediated signalling to a functionally effective level upregulates said vascular smooth muscle cell activity and downregulating sphingosine kinase mediated signalling to a functionally ineffective level downregulates said vascular smooth muscle cell activity.

In still another aspect there is provided a method of modulating bronchial smooth muscle cell activity, said method comprising modulating the functional activity of sphingosine kinase mediated signalling wherein upregulating sphingosine kinase mediated signalling to a functionally effective level upregulates said bronchial smooth muscle cell activity and downregulating sphingosine kinase mediated signalling to a functionally ineffective level downregulates said bronchial smooth muscle cell activity.

In yet another aspect the present invention provides a method of modulating smooth muscle cell tone, said method comprising modulating the functional activity of sphingosine kinase mediated signalling wherein upregulating sphingosine kinase mediated signalling to a functionally effective level upregulates said smooth muscle tone and down-regulating sphingosine kinase mediated signalling to a functionally ineffective level down-regulates said smooth muscle tone.

In yet still another aspect the present invention provides a method of modulating vascular smooth muscle cell tone, said method comprising modulating the functional activity of sphingosine kinase mediated signalling wherein upregulating sphingosine kinase mediated signalling to a functionally effective level upregulates said vascular smooth muscle tone and downregulating sphingosine kinase mediated signalling to a functionally ineffective level downregulates said vascular smooth muscle tone.

In still yet another aspect the present invention provides a method of modulating bronchial smooth muscle cell tone, said method comprising modulating the functional activity of sphingosine kinase mediated signalling wherein upregulating sphingosine kinase mediated signalling to a functionally effective level upregulates said bronchial smooth muscle tone and downregulating sphingosine kinase mediated signalling to a functionally ineffective level downregulates said bronchial smooth muscle tone.

Still another aspect of the present invention is directed to a method of regulating smooth muscle cell activity in a mammal, said method comprising modulating the functional activity of sphingosine kinase mediated signalling in said mammal wherein upregulating sphingosine kinase mediated signalling activity to a functionally effective level upregulates said smooth muscle cell activity and downregulating sphingosine kinase mediated signalling to a functionally ineffective level downregulates said smooth muscle cell activity.

Yet still another aspect of the present invention is directed to a method of regulating vascular smooth muscle cell activity in a mammal, said method comprising modulating the functional activity of sphingosine kinase mediated signalling in said mammal wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

In another aspect the present invention is directed to a method of regulating bronchial smooth muscle cell activity in a mammal, said method comprising modulating the functional activity of sphingosine kinase mediated signalling in said mammal wherein upregulating sphingosine kinase mediated signalling activity to a functionally effective level upregulates said bronchial smooth muscle cell activity and down-regulating sphingosine kinase mediated signalling to a functionally ineffective level down-regulates said bronchial smooth muscle cell activity.

In still yet another aspect there is provided a method of upregulating smooth muscle cell activity in a mammal, said method comprising administering to said mammal an effective amount of an agent for a time and under conditions sufficient to induce a functionally effective level of sphingosine kinase mediated signalling.

In a further aspect there is provided a method of upregulating smooth muscle cell activity in a mammal, said method comprising administering to said mammal an effective amount of a sphingosine kinase mediated signalling pathway component for a time and under conditions sufficient to induce a functionally effect level of sphingosine kinase mediated signalling.

In still another further aspect there is provided a method of upregulating smooth muscle cell activity in a mammal, said method comprising administering to said mammal an effective amount of a nucleotide sequence encoding a sphingosine kinase mediated signalling pathway component for a time and under conditions sufficient to induce a functionally effective level of sphingosine kinase mediated signalling.

In yet another further aspect there is provided a method of downregulating smooth muscle cell activity in a mammal, said method comprising administering to said mammal an effective amount of an agent for a time and under conditions sufficient to induce a functionally ineffective level of sphingosine kinase mediated signalling.

Still another further aspect of the present invention provides a method for the treatment and/or prophylaxis of a condition characterised by aberrant, unwanted or otherwise inappropriate smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Another aspect of the present invention provides a method for the treatment and/or prophylaxis of a condition characterised by aberrant, unwanted or otherwise inappropriate vascular smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

In another aspect the present invention contemplates a method for the treatment and/or prophylaxis of a condition characterised by aberrant, unwanted or otherwise inappropriate bronchial smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Still another aspect of the present invention relates to the use of an agent capable of modulating the functionally effective level of sphingosine kinase mediated signalling in the manufacture of a medicament for the regulation of vascular smooth muscle cell activity in a mammal wherein upregulating sphingosine kinase mediated signalling to a functionally effective level upregulates said smooth muscle cell activity and down-regulating sphingosine kinase mediated signalling to a functionally ineffective level down-regulates said smooth muscle cell activity.

In yet another aspect the present invention relates to the use of a component of the sphingosine kinase mediated signalling pathway, or a nucleic acid molecule encoding said component, in the manufacture of a medicament for the regulation of smooth muscle cell activity wherein upregulating sphingosine kinase mediated signalling to a functionally effective level upregulates said smooth muscle cell activity.

In yet still another aspect, the present invention contemplates a pharmaceutical composition comprising the modulatory agent as hereinbefore defined and one or more pharmaceutically acceptable carriers and/or diluents.

### BRIEF DESCRIPTION OF THE DRAWINGS

FIG. 1 is an image of a) Resting tone in isolated resistance arteries after selective transfection of vascular smooth muscle cells with mutants of sphingosine kinase (active: Sphk1, n=12, inactive: hSK-G82D, n−16) alone or in combination with dominant negative mutants of RhoA (N19RhoA, n=8) or Rho kinase (KD1A, n=8). L83RhoA is a constitutively active RhoA mutant. Green fluorescent protein (GFP, n=18)-transfected arteries served as controls. Transfection efficacy was controlled in separate experiments using a Sphk-GFP fusion protein cloned into the same vector (pcDNA3) as all other constructs used in this study. As shown in images c) (GFP transfection, confocal plane) and d) (Sphk-GFP, non-confocal), virtually all cells within the vascular wall of transfected resistance arteries expressed GFP and Sphk-GFP, respectively. In contrast, no fluorescence was detectable in non-transfected arteries (b).

FIG. 2 is a graphical representation of the kinetics of pressure-induced myogenic responses of isolated resistance arteries that were transfected with green fluorescent protein (GFP, n=8, a) or Sphk1 (n=12, b). Initial distensions elicited by increases in pressure from 45 mmHg to 110 mmHg were partially reversed by a subsequent vasoconstriction that was significantly accelerated and augmented in arteries overexpressing Sphk1. Enforced expression of Sphk1 did also augment pressure-induced increases in smooth muscle Ca²⁺. Data were normalised to resting Ca²⁺ and diameter levels, respectively, and summarised every 10 sec (symbols show means±SEM).

FIG. 3 is a graphical representation of pressure-induced myogenic responses in isolated resistance arteries after selective transfection of vascular smooth muscle cells with mutants of Sphk1 (active: Sphk1, n=12; dominant-negative: hSK-G82D, n=16) alone or in combination with dominant negative mutants of RhoA (N19RhoA, n=8) or Rho kinase (KD1A, n=8) genetically activated the RhoA/Rho kinase pathway. Bars depict steady state levels of reversal of initial distension (RID) after 4 min (means±SEM).

FIG. 4 is a graphical representation of the kinetics of pressure-induced increased in smooth muscle Ca²⁺ in arteries that were transfected with green fluorescent protein (GFP, n=8), sphingosine kinase (Sphk1, n=12), dominant negative sphingosine kinase (hSK-G82D, n=16) or dominant active RhoA (L63RhoA, n=8). Sphk1-transfected arteries showed an augmented increase in Ca²⁺ Activation of the RhoA pathway (L63RhoA) did not affect kinetics of pressure-induced increases in Ca²⁺. Genetic inhibition of sphingosine kinase by its dominant negative mutant hSK-G82D resulted in significantly attenuated and delayed increases in Ca²⁺. Data were normalised to resting Ca²⁺ levels and summarised every 10 sec (symbols depicting means±SEM).

FIG. 5 is a graphical representation of the repetitive stimulation of Sphk1-transfected arteries by increases in transmural pressure from 45 to 110 mmHg over 5 min intercepted by 20 min breaks progrediently increased resting tone. Displayed values (means±SEM, n=6) represent maximal diameter (max), resting tone at start (#1), and resting tone after the first (#2) and second (#3) myogenic response.

FIG. 6 is a graphical representation indicating that the constrictions of depolarized resistance arteries (120 mmol/L K⁺) in response to increases in extracellular Ca²⁺ (Ca²⁺ₑₓ from 0 to 3 mmol/L) were significantly attenuated in presence of 10 μmol/L Sodium nitroprusside (n=7). This effect was completely reversed after inhibition of the soluble guanylate cyclase with ODQ (1 μmol/L, n=7).

(mean±SEM, significant differences between groups (p<0.05) are indicated by * for SNP vs. control; and by # for ODQ vs SNP)

FIG. 7 is a graphical representation indicating that the SNP-induced decrease in contractility of depolarized resistance arteries elicited by increasing Ca²⁺ ex was reversed after inhibition of myosin light chain phosphatase by calyculin A (120 nmol/L, n=7). Resulting levels of intracellular Ca²⁺ were similar in all groups for any given Ca²⁺ₑₓ(top panel), suggesting that the reduction of contractility was due to a Ca²⁺-desensitizing mechanism induced by NO.

(mean±SEM, significant differences between groups (p<0.05) are indicated by * for SNP vs. control; and by # for calyculin A+SNP vs SNP and by § for calyculin A vs control)

FIG. 8 is an image demonstrating that the stimulation of intact resistance arteries with 100 mnol/L S1P for 2 min induced translocation of the MLCP myosin-binding subunit (MYPT1) that was cytosolic under resting conditions (“control”) to the smooth muscle cell plasmamembrane (“S1P”). Translocation was confirmed by western blotting tissue samples obtained from hamster aortae.

Translocation of MYPT1 upon stimulation with S1P was absent in N19RhoA transfected (bottom left) and Y27632-pretreated (bottom right) resistance arteries.

(each image is representative for 3-5 independent experiments, “C”=cytosolic, “P”=particulate fraction, western blot shown is representative for 3 experiments)

FIG. 9 is an image of: a) The NO-induced desensitization to Ca²⁺ in depolarized resistance arteries (120 mmol/L K⁺) was blocked after stimulation of the RhoA/Rho kinase pathway with the phospholipid mediator sphingosine-1-phosphate (S1P, 10 nmol/L, n=6). Immunostaining of RhoA revealed a cytosolic distribution in unstimulated (b)) and a translocation to the membrane in arteries stimulated with S1P (10 nmol/L, 2 min, c)) and those stimulated with 10 nmol/L S1P before 3 min exposure to 10 μmol/L SNP.

(mean±SEM, significant differences between groups (p<0.05) are indicated by * for SNP vs. SNP+S1P; and by # for S1P vs. control)

FIG. 10 is an image of: a) Specific inhibition of RhoA by transfection of a C3 transferase-encoding plasmid significantly desensitized the contractile apparatus to Ca²⁺. b) NO-induced dilations were significantly augmented in NE (0.3 μmmol/L)-preconstricted resistance arteries when RhoA/Rho kinase activity was inhibited by either Y27632 (1 μmol/L, n=5) or by overexpression of N19RhoA (n=6).

Neither N19RhoA nor Y27632 did significantly affect resting (Y27632: 198±17 vs. (control) vs. 195±18 μm (Y27632)) or NE-induced tone (109±5 vs. 115±5 μm). (mean±SEM, significant differences between groups (p<0.05) are indicated by * for Y27632 vs. control; and by # for N19RhoA-transfected arteries vs control)

c) Resistance arteries transfected with RhoA-GFP fusion protein showed expression of RhoA-GFP in virtually all VSMCs.

### DETAILED DESCRIPTION OF THE INVENTION

The present invention is predicated, in part, on the determination that the signalling pathway which regulates smooth muscle cell tone, in particular vascular smooth muscle cell tone, is mediated by sphingosine kinase. This development now permits the rational design of therapeutic and/or prophylactic methods for treating conditions characterised by aberrant or unwanted smooth muscle cell tone.

Accordingly, one aspect of the present invention is directed to a method of modulating smooth muscle cell activity, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Reference to “smooth muscle cell” should be understood as a reference to the cells of smooth muscle tissue. Without limiting the present invention to any one theory or mode of action smooth muscle is muscle tissue which generally functions in an involuntary manner and differs from striated muscle in terms of exhibiting a much higher actin:myocin ratio and the ability to contract to a much smaller fraction of its resting length. Smooth muscle cells are found in blood vessel walls, surrounding the intestine and in the uterus. The contractile system and its control resemble those of motile tissue cells such as fibroblasts and leukocytes. The phrase “smooth muscle cell” should also be understood as a reference to cells which exhibit one or more of the morphology, phenotype and/or functional activity of smooth muscle cells and is also a reference to mutants or variants thereof. “Variants” include, but are not limited to, cells exhibiting some but not all of the morphological or phenotypic features or functional activities of smooth muscle cells at any differentiative stage of development. “Mutants” include, but are not limited to, smooth muscle cells which have been naturally or non-naturally modified such as cells which are genetically modified.

It should also be understood that the smooth muscle cells of the present invention may be at any differentiative stage of development. Accordingly, the cells may be immature and therefore functionally incompetent in the absence of further differentiation. In this regard, highly immature cells, such as stem cells, which retain the capacity to differentiate into smooth muscle cells, should nevertheless be understood to satisfy the definition of “smooth muscle cells” as utilised herein due to their capacity to differentiate into smooth muscle cells under appropriate conditions. Preferably, the subject smooth muscle cell is vascular, gastric, bladder, intestinal, bronchial or uterine smooth muscle cell.

Most preferably, said smooth muscle cell is a vascular, bronchial or intestinal smooth muscle cell.

Accordingly, in one preferred embodiment there is more particularly provided a method of modulating vascular smooth muscle cell activity, said method comprising modulating the functional activity of sphingosine kinase mediated signalling wherein upregulating sphingosine kinase mediated signalling to a functionally effective level upregulates said vascular smooth muscle cell activity and downregulating sphingosine kinase mediated signalling to a functionally ineffective level downregulates said vascular smooth muscle cell activity.

In another preferred embodiment there is provided a method of modulating bronchial smooth muscle cell activity, said method comprising modulating the functional activity of sphingosine kinase mediated signalling wherein upregulating sphingosine kinase mediated signalling to a functionally effective level upregulates said bronchial smooth muscle cell activity and downregulating sphingosine kinase mediated signalling to a functionally ineffective level downregulates said bronchial smooth muscle cell activity.

Reference to smooth muscle cell “activity” should be understood as a reference to any one or more of the functional activities which a smooth muscle cell is capable of performing, for example, maintenance of vascular smooth muscle cell tone. By “tone” is meant the contractile status of a smooth muscle cell. In this regard, without limiting the present invention to any one theory or mode of action, the sometimes severe breathing difficulties associated with conditions such as asthma result from the unwanted constriction of the bronchial smooth muscle cells. This constriction can be brought on by any one of a number of causes, although an unwanted immune response to an allergen (often an apparently innocuous one) is one of the most common causes. Such allergens include, but are not limited to, house dust mites, foods (e.g. nuts), latex, drugs (e.g. penicillin), pet fur, chemicals, or toxins (e.g. bee sting). Although an unwanted immunological response can manifest in different ways, for example runny eyes or nose or sneezing, it is the responses which lead to varying degrees of breathing difficulties (e.g. coughing, wheezing and shortness of breath) which can present a potentially life threatening situation. In such conditions, whether they be classified as asthma, an “allergy” (e.g. pet or food allergy) or anaphylaxis, the breathing difficulties which are observed are due to constriction of the bronchial smooth muscle cells due to an unwanted immune response. However, it should also be understood that there are a number of conditions which similarly result in bronchial smooth muscle constriction, but which conditions are not associated with an immunological cause. For example, an anaphylactoid reaction mimics anaphylactic shock but is not an immunological disorder. The treatment of the bronchial constriction symptoms of any one or more of these conditions would benefit from a means of releasing constriction of the bronchial smooth muscle cells.

Still without limiting the present invention in any way, and in the context of another of the preferred embodiments of the present invention, the aorta and other systemic arteries are surrounded by smooth muscle which, via contraction and relaxation, can alter the radius of the arteries. This enables the arteries to function as a pressure reservoir for maintaining blood flow through the tissues. Further, it provides a means of altering the resistance to blood flow, thereby effectively providing a means of altering blood pressure. Specifically, as the radius of an artery is decreased (due to constriction of the smooth muscle cells) the resistance within the artery to blood flow markedly increases. The converse is true in relation to relaxation of the smooth muscle around an artery. For example, doubling the radius of an artery, via relaxation of the smooth muscle, would decrease the resistance it provides approximately 16-fold and therefore increase blood flow through it 16-fold. At any given point in time, the vascular smooth muscle will exhibit a certain basal level of constriction (often referred to as “resting tone”).

Arterioles are primarily responsible for determining the relative blood flow distribution to different organs. Since the driving pressure is identical for each arteriole, differences in flow are generally determined by differences in the resistance to flow offered by each arteriole. Since the length of the arterioles are approximately the same and the viscosity of the blood remains relatively constant, differences in resistance offered by the arterioles are due largely to differences in their radii. The arterioles comprise smooth muscle which can relax or contract (ie. modulation of smooth muscle cell “tone”) thereby changing the radius of the lumen of the arteriole. Accordingly, the pattern of blood flow distribution will largely depend upon the degree of arteriolar smooth muscle constriction within each organ and tissue. The smooth muscles surrounding arterioles are mainly single unit smooth muscle and possess a large degree of inherent myogenic activity ie. “spontaneous” constrictions. This tone is responsible for a large proportion of the basal resistance offered by the arterioles. However, a variety of physiological factors act upon the smooth muscle by altering the intracellular calcium concentration to either increase or decrease the degree of constriction, thereby altering the vessel's resistance. The controlling mechanism, in a normal physiological situation, will fall into one of two general categories being local controls or extrinsic controls. Local controls include, for example, active hyperaemia and pressure auto regulation (also known as pressure-induced myogenic vasoconstriction). Extrinsic controls include those provided by the sympathetic nerves and hormones. In addition to modulating intracellular calcium levels, it has also been determined that smooth muscle tone is regulated by the modulation of myofilament calcium sensitivity.

Accordingly, the present invention still more particularly provides a method of modulating smooth muscle cell tone, said method comprising modulating the functional activity of sphingosine kinase mediated signalling wherein upregulating sphingosine kinase mediated signalling to a functionally effective level upregulates said smooth muscle tone and downregulating sphingosine kinase mediated signalling to a functionally ineffective level downregulates said smooth muscle tone.

Preferably, said smooth muscle is vascular, bronchial, gastric, bladder, intestinal or uterine smooth muscle.

Accordingly, in one preferred embodiment the present invention provides a method of modulating vascular smooth muscle cell tone, said method comprising modulating the functional activity of sphingosine kinase mediated signalling wherein upregulating sphingosine kinase mediated signalling to a functionally effective level upregulates said vascular smooth muscle tone and downregulating sphingosine kinase mediated signalling to a functionally ineffective level downregulates said vascular smooth muscle tone.

In another preferred embodiment the present invention provides a method of modulating bronchial smooth muscle cell tone, said method comprising modulating the functional activity of sphingosine kinase mediated signalling wherein upregulating sphingosine kinase mediated signalling to a functionally effective level upregulates said bronchial smooth muscle tone and downregulating sphingosine kinase mediated signalling to a functionally ineffective level downregulates said bronchial smooth muscle tone.

Reference to “sphingosine kinase mediated signalling” should be understood as a reference to an intracellular signalling pathway which utilises one or both of sphingosine kinase and/or sphingosine-1-phosphate or functional derivatives of homologues thereof. Sphingosine kinase is a key regulatory enzyme in the activity of the sphingosine kinase signalling pathway and functions to generate the endogenous sphingolipid mediator sphingosine-1-phosphate. Still further, and without limiting the present invention in any way, sphingosine kinase and sphingosine-1-phosphate are thought to be part of a signalling cascade which activates the RhoA/Rho kinase pathway to lead to modulation of smooth muscle cell tone, in particular arterial vascular smooth muscle cell tone.

Reference to “modulating” should be understood as a reference to upregulating or downregulating the subject smooth muscle cell activity. Reference to “downregulating” smooth muscle cell activity should therefore be understood as a reference to preventing, reducing (eg. slowing) or otherwise inhibiting one or more aspects of the functioning of the smooth muscle cell (for example retarding or preventing arterial constriction) while reference to “upregulating” should be understood to have the converse meaning.

Reference to sphingosine kinase mediated signalling “activity” should be understood as a reference to any one or more of the activities which the sphingosine kinase mediated signalling pathway can perform. For example, and without limiting the present invention in any way, in accordance with one of the preferred embodiments, myogenic vasoconstriction is controlled by the activity of RhoA and Rho kinase under the overarching signalling control of sphingosine kinase dependent, and therefore sphingosine-1-phosphate mediated, activation of the RhoA/Rho kinase pathway. It has been determined that this is an integral component of the myogenic response. Activation of the RhoA/Rho kinase pathway by mechanisms other then sphingosine kinase/sphingosine-1-phosphate mediated signalling results in the induction of significantly smaller myogenic responses. In this regard, it should be understood that the subject sphingosine kinase and/or sphingosine-1-phosphate may function directly or indirectly to modulate smooth muscle cell activity. By “indirect” modulation is meant that the sphingosine kinase and/or sphingosine-1-phosphate do not directly act to modulate smooth muscle cell tone but function via an intermediate mechanism such as the RhoA/Rho kinase signalling mechanism. However, it should be understood that the subject sphingosine kinase/sphingosine-1-phosphate may also act directly to modulate smooth muscle cell tone such as by delivering a signal directly to the contractile apparatus, ie. without involving non-sphingosine kinase pathway molecules, in order to modulate smooth muscle cell tone. In another example, upon activation the sphingosine kinase releases S1P to the extracellular space where it binds to S1P receptors in an autocrine and/or paracrine fashion. Some of these receptors (e.g. S1PR₂, which is present in resistance arteries) are linked to the RhoA/Rho kinase pathway. A further pathway is the intracellular release of Ca²⁺ from intracellular stores by S1P. This mechanism has been shown to be distinct from IP₃. This is a RhoA-independent pathway that results in modulation of contractility via activation of the myosin light chain kinase (MLCK). Accordingly, modulation of the “activity” of sphingosine kinase mediated signalling should be understood as reference to either upregulating or downregulating the signalling mechanism. Such modulation may be achieved by any suitable means and includes:
(i) modulating absolute levels of the components of the sphingosine kinase mediated signalling pathway, such as sphingosine kinase and/or sphingosine-1-phosphate, such that either more or less of these molecules are available for activation and/or to interact with downstream targets.
(ii) agonising or antagonising the components of the sphingosine kinase mediated signalling pathway, such as sphingosine kinase and/or sphingosine-1-phosphate, such that the functional effectiveness of any one or more of these molecules is either increased or decreased. For example, increasing the half life of sphingosine kinase may achieve an increase in the overall level of sphingosine kinase activity without actually necessitating an increase in the absolute intracellular concentration of sphingosine kinase. Similarly, the partial antagonism of sphingosine kinase or sphingosine-1-phosphate, for example by coupling these molecules to components that introduce some steric hindrance in relation to their binding to downstream targets, may act to reduce, although not necessarily eliminate, the effectiveness of the signalling which they provide. Accordingly, this may provide a means of downregulating sphingosine kinase mediated signalling without necessarily downregulating the absolute concentrations of the components of this pathway.

In terms of achieving the up or downregulation of sphingosine kinase mediated signalling, means for achieving this objective would be well known to the person of skill in the art and include, but are not limited to:
(i) introducing into a cell a nucleic acid molecule encoding a sphingosine kinase signalling pathway component or functional equivalent, derivative or analogue thereof in order to upregulate the capacity of said cell to express the sphingosine kinase mediated pathway component;
(ii) introducing into a cell a proteinaceous or non-proteinaceous molecule which modulates transcriptional and/or translational regulation of a gene, wherein this gene may be any sphingosine kinase signalling pathway component, in particular sphingosine kinase or sphingosine-1-phosphate or functional portion thereof, or some other gene which directly or indirectly modulates the expression of the components of sphingosine kinase mediated signalling pathways;
(iii) introducing into a cell one or more of the sphingosine kinase mediated signalling pathway component expression products (in either active or inactive form) or a functional derivative, homologue, analogue, equivalent or mimetic thereof;
(iv) introducing a proteinaceous or non-proteinaceous molecule which functions as an antagonist to any one or more components of the sphingosine kinase signalling pathway expression product;
(v) introducing a proteinaceous or non-proteinaceous molecule which functions as an agonist of the sphingosine kinase mediated signalling pathway expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the sphingosine kinase signalling pathway components or small molecules capable of acting as agonists or antagonists.

Chemical agonists may not necessarily be derived from the components of the sphingosine kinase mediated signalling pathway product but may share certain conformational similarities. Alternatively, chemical agonists may be specifically designed to meet certain physiochemical properties. Antagonists may be any compound capable of blocking, inhibiting or otherwise preventing components of the sphingosine kinase mediated signalling pathway from carrying out their normal biological function, such as molecules which prevent activation or else prevent the downstream functioning of activated molecules. Antagonists include monoclonal antibodies and antisense nucleic acids which prevent transcription or translation of the genes or mRNA of components of the sphingosine kinase mediated signalling pathway in mammalian cells. Modulation of expression may also be achieved utilising antigens, RNA, ribosomes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the sphingosine kinase gene (or any other gene which encodes a component of the sphingosine kinase signalling pathway) or functional equivalent or derivative thereof with an agent and screening for the modulation of sphingosine kinase protein production or functional activity, modulation of the expression of a nucleic acid molecule encoding sphingosine kinase or modulation of the activity or expression of a downstream sphingosine kinase cellular target. Detecting such modulation can be achieved utilising techniques such as Western blotting, electrophoretic mobility shift assays and/or the readout of reporters of sphingosine kinase activity such as luciferases, CAT and the like.

It should be understood that the sphingosine kinase gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate sphingosine kinase activity, at either the nucleic acid or expression product levels, or the gene may require activation—thereby providing a model useful for, inter alia, screening for agents which up regulate sphingosine kinase expression. Further, to the extent that a sphingosine kinase nucleic acid molecule is transfected into a cell, that molecule may comprise the entire sphingosine kinase gene or it may merely comprise a portion of the gene such as the portion which regulates expression of the sphingosine kinase product. For example, the sphingosine kinase promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream sphingosine kinase regulatory target (for example, sphingosine-1-phosphate), rather than sphingosine kinase itself. Yet another example includes sphingosine kinase binding sites ligated to a minimal reporter. For example, modulation of sphingosine kinase activity can be detected by screening for the modulation of the functional activity of a smooth muscle cell. This is an example of an indirect system where modulation of sphingosine kinase expression, per se, is not the subject of detection. Rather, modulation of the molecules which sphingosine kinase regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the sphingosine kinase nucleic acid molecule or expression product itself or which modulate the expression of an upstream molecule, which upstream molecule subsequently modulates sphingosine kinase expression or expression product activity. Accordingly, these methods provide a mechanism of detecting agents which either directly or indirectly modulate sphingosine kinase expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of sphingosine kinase or the activity of the sphingosine kinase expression product. Said molecule acts directly if it associates with the sphingosine kinase nucleic acid molecule or expression product to modulate expression or activity, respectively. Said molecule acts indirectly if it associates with a molecule other than the sphingosine kinase nucleic acid molecule or expression product which other molecule either directly or indirectly modulates the expression or activity of the sphingosine kinase nucleic acid molecule or expression product, respectively. Accordingly, the method of the present invention encompasses the regulation of sphingosine kinase nucleic acid molecule expression or expression product activity via the induction of a cascade of regulatory steps.

The term “expression” refers to the transcription and translation of a nucleic acid molecule. Reference to “expression product” is a reference to the product produced from the transcription and translation of a nucleic acid molecule. Reference to “modulation” should be understood as a reference to upregulation or downregulation.

“Derivatives” of the molecules herein described (for example sphingosine kinase, sphingosine-1-phosphate or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, sphingosine kinase or derivative thereof may be fused to a molecule to facilitate its entry into a cell. Analogs of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogs.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” of sphingosine kinase or sphingosine-1-phosphate should be understood to mean molecules which exhibit at least some of the functional activity of the form of sphingosine kinase or sphingosine-1-phosphate of which it is a variant. A variation may take any form and may be naturally or non-naturally occurring. A mutant molecule is one which exhibits modified functional activity.

By “homologue” is meant that the molecule is derived from a species other than that which is being treated in accordance with the method of the present invention. This may occur, for example, where it is determined that a species other than that which is being treated produces a form of sphingosine kinase or sphingosine-1-phosphate which exhibits similar and suitable functional characteristics to that of the sphingosine kinase or sphingosine-1-phosphate which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing sphingosine kinase and/or sphingosine-1-phosphate analogues which exhibit properties such as more potent pharmacological effects. Sphingosine kinase and/or sphingosine-1-phosphate or a functional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of sphingosine kinase and/or sphingosine-1-phosphate, it may also be desirable to identify and utilise molecules which function agonistically or antagonistically to sphingosine kinase and/or sphingosine-1-phosphate in order to up or downregulate the functional activity of sphingosine kinase and/or sphingosine-1-phosphate in relation to modulating smooth muscle cell activity. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of sphingosine kinase and/or sphingosine-1-phosphate capable of acting as agonists or antagonists. Chemical agonists may not necessarily be derived from sphingosine kinase and/or sphingosine-1-phosphate but may share certain conformational similarities. Alternatively, chemical agonists may be specifically designed to mimic certain physiochemical properties of sphingosine kinase and/or sphingosine-1-phosphate.

Antagonists may be any compound capable of blocking, inhibiting or otherwise preventing sphingosine kinase and/or sphingosine-1-phosphate from carrying out its normal biological functions. Antagonists include monoclonal antibodies specific for sphingosine kinase and/or sphingosine-1-phosphate or parts of sphingosine kinase and/or sphingosine-1-phosphate.

Analogues of sphingosine kinase and/or sphingosine-1-phosphate or of sphingosine kinase and/or sphingosine-1-phosphate agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional amino acid | Code |
|---|---|
| α-aminobutyric acid | Abu |
| α-amino-α-methylbutyrate | Mgabu |
| aminocyclopropanecarboxylate | Cpro |
| aminoisobutyric acid | Aib |
| aminonorbornylcarboxylate | Norb |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| a-methyl-aminoisobutyrate | Maib |
| α-methyl- -aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

Reference herein to attaining either a “functionally effective level” or “functionally ineffective level” of sphingosine kinase mediated signalling should be understood as a reference to attaining that level of signalling at which modulation of smooth muscle cell activity, in particular vascular or bronchial smooth muscle cell tone, can be achieved, whether that be upregulation or downregulation. In this regard, it is within the skill of the person of skill in the art to determine, utilising routine procedures, the threshold level of signalling above which smooth muscle cell activity can be upregulated and below which smooth muscle cell activity is downregulated. For example, suitable for use in this regard is any method which regulates the phosphorylation status or the cellular localisation of sphingosine kinase, as would any method which is based on the alteration of RNA synthesis of sphingosine kinase (for example, antisense constructs, DNAzymes or RNAi could change the levels of proteins). It should be understood that reference to an “effective level” means the level necessary to at least partly attain the desired response. The amount will vary depending on the health and physical condition of the cellular population and/or individual being treated, the taxonomic group of the cellular population and/or individual being treated, the degree of up or downregulation which is desired, the formulation of the composition which is utilised, the assessment of the medical situation and other relevant factors. Accordingly, it is expected that this level may vary between individual situations, thereby falling in a broad range, which can be determined through routine trials.

Without limiting the present invention to any one theory or mode of action, exemplification in the context of one of the preferred embodiments has determined that sphingosine kinase-1 phosphorylates sphingosine to create elevated levels of sphingosine-1-phosphate thereby increasing the calcium sensitivity of vascular smooth muscle. However, in addition to leading to the sensitisation of the contractile apparatus to calcium, the sphingosine kinase signalling pathway also leads to increases in intracellular calcium concentration which is the starting signal for the myogenic response. This effect shows that the sphingosine kinase is a fast responding system and involved in regulation of both main pathways that can induce vasoconstriction, namely the Ca²⁺-dependent and the RhoA-mediated Ca²⁺-independent. This is a unique situation which renders sphingosine kinase such a potent vasoconstrictor. It has still further been determined that these pathways are not separately activated but are simultaneously initiated by sphingosine-1-phosphate in a precise spatial temporal interaction. In particular, sphingosine-1-phosphate augments pressure induced myogenic constriction in arteries via the RhoA/Rho kinase pathway. However, sphingosine-1-phosphate augments smooth muscle's calcium sensitisation in a Rho independent manner. Accordingly, both the establishment and maintenance of resting tone and the degree of myogenic response which occurs subsequently to stimulation, whether that be pressure induced stimulation or stimulation by some other means (such as hormonal) can be modulated by altering the expression and activity of sphingosine kinase. Accordingly this molecule is a major determinant of vascular tone both directly and via the RhoA/Rho family of molecules.

The method of the present invention contemplates the modulation of smooth muscle cell functioning both in vitro and in vivo. Although the preferred method is to treat an individual in vivo it should nevertheless be understood that it may be desirable that the method of the invention may be applied in an in vitro environment, for example to provide an in vitro model of vascular smooth muscle cell tone analysis. In another example the application of the method of the present invention in an in vitro environment may extend to providing a readout mechanism for screening technologies such as those hereinbefore described. That is, molecules identified utilising these screening techniques can be assayed to observe the extent and/or nature of their functional effect on smooth muscle cells which have been functionally modulated according to the method of the present invention.

Although the preferred method is to downregulate smooth muscle cell tone, for example downregulating arterial resistance (for example in order to downregulate the progression of hypertension or to encourage greater blood flow to tissues) or bronchial constriction, it should be understood that there may also be circumstances in which it is desirable to upregulate smooth muscle cell tone. For example, hypotension is one example where it would be desirable to increase vascular tone. So too is it desirable in septic shock. Without limiting the present invention in any way, patients with a septic shock suffer maximal peripheral vasodilation because of massive smooth muscle iNOS (inducible NO synthase) induction with an NO output that exceeds normal rates by at least the factor 1000. In order to maintain blood pressure cardiac output is dramatically increased. This constellation mostly concludes fatally for the patient (especially in children in a terminal stage of bacterial meningitis, Waterhouse-Friedrichsen-syndrome). A strategy to interfere with the massive NO production is to apply NOS inhibitors. Unfortunately, these inhibitors are still not specific enough to solely inhibit iNOS but also inhibit the important endothelial isoform (eNOS). Therefore, these strategies lead to the complete opposite of the initial pathophysiological state, namely an increase in peripheral resistance that results in a hypertensive crisis. For this form of shock it is desirable to use a vasoconstrictor that prevents NO-dependent dilations in resistance arteries, such as that provided by the present invention.

Accordingly, another aspect of the present invention is directed to a method of regulating smooth muscle cell activity in a mammal, said method comprising modulating the functional activity of sphingosine kinase mediated signalling in said mammal wherein upregulating sphingosine kinase mediated signalling activity to a functionally effective level upregulates said smooth muscle cell activity and downregulating sphingosine kinase mediated signalling to a functionally ineffective level downregulates said smooth muscle cell activity.

Preferably, said smooth muscle is vascular, bronchial, gastric, intestinal or uterine smooth muscle.

More particularly, the present invention is directed to a method of regulating vascular smooth muscle cell activity in a mammal, said method comprising modulating the functional activity of sphingosine kinase mediated signalling in said mammal wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

In another preferred embodiment the present invention is directed to a method of regulating bronchial smooth muscle cell activity in a mammal, said method comprising modulating the functional activity of sphingosine kinase mediated signalling in said mammal wherein upregulating sphingosine kinase mediated signalling activity to a functionally effective level upregulates said bronchial smooth muscle cell activity and downregulating sphingosine kinase mediated signalling to a functionally ineffective level downregulates said bronchial smooth muscle cell activity.

Preferably, said smooth muscle cell activity is smooth muscle cell tone.

Modulation of said sphingosine kinase mediated signalling activity is achieved by the administration of a component of said sphingosine kinase mediated signalling pathway, a nucleic acid molecule encoding a component of said sphingosine kinase mediated signalling pathway or an agent which effects modulation of any one or more of said component's functional activity or expression of genes encoding said component (herein collectively referred to as “modulatory agents”).

Accordingly, in one preferred embodiment there is provided a method of upregulating smooth muscle cell activity in a mammal, said method comprising administering to said mammal an effective amount of an agent for a time and under conditions sufficient to induce a functionally effective level of sphingosine kinase mediated signalling.

In another preferred embodiment there is provided a method of upregulating smooth muscle cell activity in a mammal, said method comprising administering to said mammal an effective amount of a sphingosine kinase mediated signalling pathway component for a time and under conditions sufficient to induce a functionally effective level of sphingosine kinase mediated signalling.

In still another preferred embodiment there is provided a method of upregulating smooth muscle cell activity in a mammal, said method comprising administering to said mammal an effective amount of a nucleotide sequence encoding a sphingosine kinase mediated signalling pathway component for a time and under conditions sufficient to induce a functionally effective level of sphingosine kinase mediated signalling.

In yet another preferred embodiment there is provided a method of downregulating smooth muscle cell activity in a mammal, said method comprising administering to said mammal an effective amount of an agent for a time and under conditions sufficient to induce a functionally ineffective level of sphingosine kinase mediated signalling.

In accordance with these preferred embodiments, said smooth muscle cell activity if preferably vascular smooth muscle cell tone.

In still another preferred embodiment, said smooth muscle is vascular, bronchial, gastric, bladder, intestinal or uterine smooth muscle.

Most preferably, said smooth muscle is vascular or bronchial.

Reference to “induce” should be understood as a reference to achieving the desired sphingosine kinase mediated signalling level, whether that be a functionally effective level or a functionally ineffective level. Said induction is most likely to be achieved by the upregulation or downregulation of the functional activity of one or more components of the sphingosine kinase mediated signalling pathway, as hereinbefore described, although any other suitable means of achieving induction are nevertheless herewith encompassed by the method of the present invention.

A further aspect of the present invention relates to the use of the invention in relation to the treatment and/or prophylaxis of disease conditions or other unwanted conditions. Without limiting the present invention to any one theory or mode of action, the regulation of smooth muscle cell activity, and in particular vascular or bronchial smooth muscle cell tone, is an essential requirement in terms of controlling blood pressure and breathing, respectively.

The present invention therefore contemplates a method for the treatment and/or prophylaxis of a condition characterised by aberrant, unwanted or otherwise inappropriate smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Preferably, said smooth muscle is vascular, bronchial, gastric, bladder, intestinal or uterine smooth muscle.

Most preferably, said smooth muscle is vascular or bronchial.

More preferably, said smooth muscle cell activity is smooth muscle cell tone.

Reference to “aberrant, unwanted or otherwise inappropriate” smooth muscle cell activity should be understood as a reference to underactive functioning, to physiologically normal functioning which is inappropriate in that it is unwanted or to overactive smooth muscle cell functioning. As detailed hereinbefore, there are a number of conditions which are dependent on the induction of the correct level of smooth muscle cell functioning, and in particular smooth muscle cell tone. For instance, and in relation to the preferred embodiments disclosed herein, in individuals experiencing hypertension, the downregulation of sphingosine kinase mediated signalling in vascular smooth muscle cells provides a means of decreasing arterial resistance and thereby decreasing the individual's blood pressure. In the context of unwanted bronchial constriction (for example as induced by asthma or anaphylactic shock), downregulation of sphingosine kinase mediated signalling in the bronchial smooth muscle cells provides a means of dilating the bronchial passages and thereby easing breathing difficulties.

The present invention therefore preferably contemplates a method for the treatment and/or prophylaxis of a condition characterised by aberrant, unwanted or otherwise inappropriate vascular smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Preferably, said vascular smooth muscle cell activity is vascular smooth muscle cell tone. Most preferably, said condition is hypertension and smooth muscle cell constriction is relaxed via downregulation of sphingosine kinase mediated signalling.

In another preferred embodiment the present invention contemplates a method for the treatment and/or prophylaxis of a condition characterised by aberrant, unwanted or otherwise inappropriate bronchial smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

Preferably, said bronchial smooth muscle cell activity is bronchial smooth muscle cell tone. Most preferably, said condition is asthma and smooth muscle cell constriction is relaxed via downregulation of sphingosine kinase mediated signalling.

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day.

Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject sphingosine kinase or sphingosine-1-phosphate may be administered together with an agonistic agent in order to enhance its effects. Alternatively, in the case of hypertension, for example, a sphingosine kinase and/or sphingosine-1-phosphate antagonist may be administered together with other hypertension drugs. By “sequential” administration is meant a time difference of from seconds, minutes, hours or days between the administration of the two types of molecules. These molecules may be administered in any order.

Another aspect of the present invention relates to the use of an agent capable of modulating the functionally effective level of sphingosine kinase mediated signalling in the manufacture of a medicament for the regulation of smooth muscle cell activity in a mammal wherein upregulating sphingosine kinase mediated signalling to a functionally effective level upregulates said vascular smooth muscle cell activity and down-regulating sphingosine kinase mediated signalling to a functionally ineffective level down-regulates said smooth muscle cell activity.

In another aspect the present invention relates to the use of a component of the sphingosine kinase mediated signalling pathway, or a nucleic acid molecule encoding said component, in the manufacture of a medicament for the regulation of smooth muscle cell activity wherein upregulating sphingosine kinase mediated signalling to a functionally effective level upregulates said smooth muscle cell activity.

Preferably, said smooth muscle is vascular, bronchial, gastric, bladder, intestinal or uterine smooth muscle.

More preferably, where the subject smooth muscle is vascular smooth muscle, said activity is tone. Most preferably, said activity is constriction which is down-regulated.

In another preferred embodiment, where said smooth muscle is bronchial smooth muscle, said activity is tone. Most preferably, said activity is constriction which is down-regulated.

The term “mammal” and “subject” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding sphingosine kinase or a modulatory agent as hereinbefore defined. The vector may, for example, be a viral vector.

The present invention is further defined by the following non-limiting Examples.

### EXAMPLE 1

### Sphingosine Kinase Modulates Microvascular Tone and Myogenic Responses via Activation of RhoA/Rho Kinase

Methods and Materials

Isolation of Resistance Arteries and Smooth Muscle Transfection in Artery Culture

The preparation of the vessels, the technique of calcium and diameter measurements (Bolz, S. S., de Wit, C., Pohl, U. (1999) Br J Pharmacol 128:124-134) as well as the artery culture and transfection method (Bolz, S. S., Pieperhoff, S., de Wit, C. et al. (2000) Am J Physiol Heart Circ Physiol 279:H1434-H1439) were previously described in more detail. Briefly, segments of small resistance arteries (maximal diameter 213±3 μm, n+116) were excised from the gracilis muscle of female syrian hamsters, cannulated with glass micropipettes and perfused with culture medium at a transmural pressure of 45 mmHg. for selective transfection of smooth muscle cells, 60 μl/mL transfection reagent (Effectene, Qiagen, Germany) and 5 μg of the respective DNA plasmid were added to the organ bath for 20-22 h.

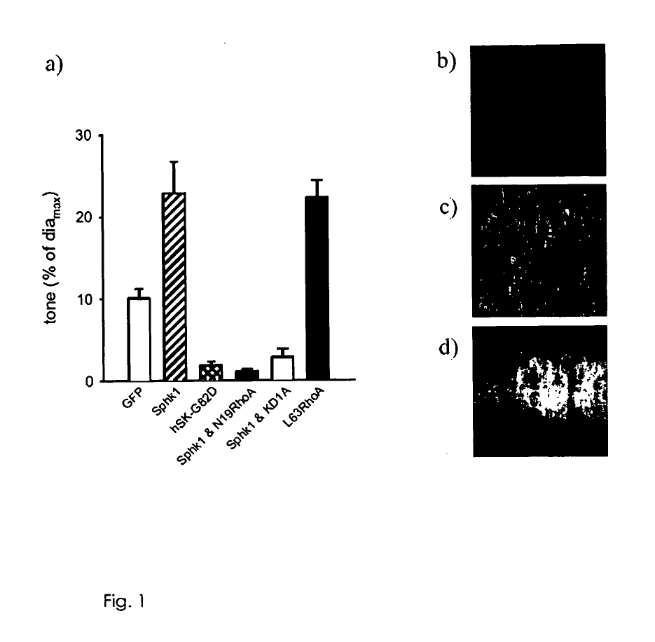

Transfection efficacy was assessed using plasmids coding for GFP or a Sphk-GFP fusion protein (FIG. 1).

Plasmids

The plasmids encoding human Sphk1 and its dominant negative mutant hSK-G82D were described previously (Pitson, S. M., Moretti, P. A., Zebol, J. R. et al. (2000) J Biol Chem 275:33945-33950). The RhoA mutants N19RhoA and L63RhoA were a kind gift by Dr. Alan Hall, Medical Research Council Laboratory for Molecular Cell Biology, University College London, UK, the plasmid coding for dominant negative Rho kinase was provided by Dr. Shuh Narumiya, Dept. of Pharmacology, Kyoto University Faculty of Medicine, Sakyo, Kyoto, Japan.

Ca²⁺ and Diameter Measurements in Genetically Modified Resistance Arteries

After termination of the culture period resistance arteries were washed with MOPS-buffered salt solution and transferred to an inverted microscope. Incubation with fura 2 (2 h at 37° C.) from the abluminal side allowed selective determination of smooth muscle Ca²⁺ within the vascular wall. Simultaneous measurements of smooth muscle intracellular Ca²⁺ and diameter were performed as described previously (Bolz, S. S., de Wit, C., Pohl, U. (1999) supra). In all 116 arteries included in the study, smooth muscle and endothelial function was assessed by testing all resistance arteries for their constrictor response to norepinephrine (NE 0.3 μmol/L) and their dilator response to acetylcholine (ACh, 1 μmol/L). Arteries that did not show maximal dilations upon ACh were excluded from the study (5 out of 121 vessels). Measurements were performed at 37° C.

Statistics

Tone was calculated as % of maximal diameter [tone(% of diaₘₐₓ)=((diaₘₐₓ−diaᵣₑₛₜ)/diaₘₐₓ)×100] that was determined at the end of every experiment in Ca²⁺-free solution under stimulation with 1 μmol/L ACh.

Myogenic responses were described as % reversal of initial distension (RID) calculated as RID=((dia_{distended}−diaₜᵢₘₑ)/(dia_{distended}−diaᵣₑₛₜ))×100, with diaₜᵢₘₑ being the actual diameter of the artery at a given time point after increase in transmural pressure.

Graphic data displayed as changes in smooth muscle Ca²⁺ or diameter under pressure were described as % change from resting Ca²⁺ or diameter, respectively [for Ca²⁺:ΔCa²⁺ (% of Ca²⁺ᵣₑₛₜ)=((Ca²⁺ₚᵣₑₛₛᵤᵣₑ−Ca²⁺ᵣₑₛₜ)/Ca²⁺ᵣₑₛₜ)×100−100].

Student's t-test was used to compare steady state values, differences were considered to be significant at error probabilities les than 0.05 (p<0.05).

To compare Ca²⁺ᵢ and diameter changes over time after application of pressure, a non-linear regression analysis was employed. Briefly, the goodness of the fit to a Gompertz function was calculated for every individual curve at first and then for pooled data sets. Curves were considered to be significantly different if the F-test indicated a significantly smaller sum of squares for the deviations in each individual fit as compared to the deviation in the fit to the pooled data (Motulsky, H. J., Ransnas, L. A. (1987) FASEB J. 1:365-374).

Results

Overexpression of Sphk1 Increases Resting Tone in Resistance Arteries

Arteries transfected with green fluorescent protein (GFP, FIG. 1c) or the fusion protein Sphk-GFP (FIG. 1d) showed protein expression in virtually all smooth muscle cells of the vascular wall (FIG. 1). NE (03. μmol/L)-induced constrictions by 51±3 and 49±4% of max. diameter (OMD), respectively, and complete dilations following 1 μmol/L ACh reveated intact contractile and endothelial function in these arteries.

However, resistance arteries transfected with Sphk1 developed significantly higher resting tone (23±3% OMD, MD: 230±6 μm, n=12, FIG. 1) than arteries transfected with GFP (10±1% OMD, MD; 228±7 μm, n=18, FIG. 1), although levels of intracellular Ca²⁺ in both groups were not different.

To block endogenous generation of S1P, arteries were transfected with dominant negative Sphk1 mutant hSK-G82D, which has previously been shown to inhibit agonist-stimulated S1P generation (Pitson, S. M., Moretti, P. A., Zebol, J. R. et al. (2000) supra). This genetic manipulation resulted in an almost complete loss of resting tone (2% OMD, MD: 247±5 μm, n=16, FIG. 1) further supporting the notion that Sphk1 lays a pivotal physiological role as a determinant of microvascular resting tone.

Effects on Resting Microvascular Tone are Mediated by RhoA/Rho Kinase

To study a possible involvement of the RhoA/Rho kinase pathway, Sphk1 was coexpressed with dominant negative mutants of RhoA (N19RhoA) or Rho kinase (KD1A), respectively, to achieve highly specific inhibition of the Rho pathway. Resting tone was almost abolished in resistance arteries in which Sphk1 was coexpressed with N19RhoA (Sphk1+N19RhoA: 1.0±0.3% OMD, MD: 242±9 μm, n=8, FIG. 1) or KD1A (Sphk1+KD1A: 2.8±1.0% OMD, MD: 239±i μm, n=8, FIG. 1).

Moreover, the dominant active RhoA mutant L63RhoA (increase in tone by 22±2% OMD, MD: 223±8 μm, n=8 FIG. 1) virtually mimicked the tone-increasing effect of Sphk1.

None of the genetic manipulations affected resting intracellular Ca²⁺ levels.

Role of RhoA/Rho Kinase for the Myogenic Response in Resistance Arteries

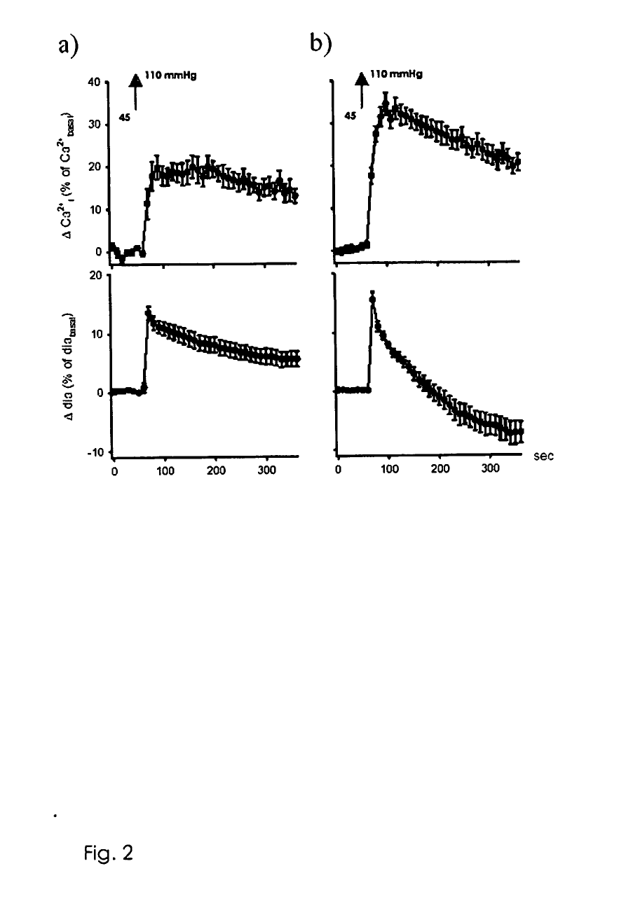

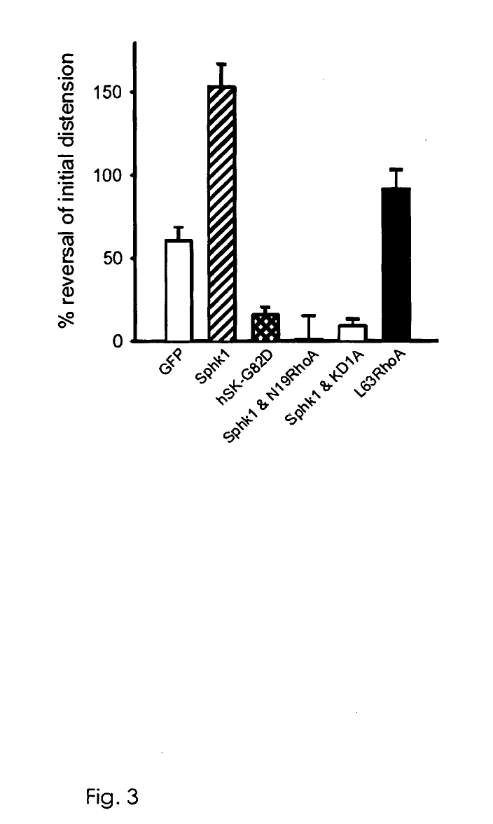

The pronounced Ca²⁺ sensitising effects we have previously demonstrated for RhoA/Rho kinase in resistance arteries (Bolz, S. S., Galle, J., Derwand, R. et al. (2000) Circulation 102:2402-2410) led us to hypothesise that activation of the RhoA pathway might also contribute to the initiation and development of the myogenic response. In fact, myogenic responses in GFP-transfected arteries (59±9% reversal of initial distension ((RID), n=8, FIGS. 2a and 3) that were comparable to those in freshly isolated or cultured arteries (Bolz, S. S., Pieperhoff, S., de Wit, C. et al. (2000) supra) were virtually abolished in resistance arteries overexpressing dominant negative mutants of RhoA (N19RhoA, 2±11% further distension, n=7) or Rho kinase (KD1A, 4±5% RID, n=6), or treated with the pharmacological Rho kinase inhibitor Y27632 (1±2% RID, n=10). Inhibition of the Rhoa/Rho kinase pathway by these agents did not affect the pressure-induced increases in Ca²⁺ (N19RhoA: 16±3%, KD1A: 17±2%, Y27632: 19±3%). These pressure-induced increases in Ca²⁺ as well as subsequent constrictions in control arteries were, however completed inhibited by the L-type calcium channel blocker felodipine (1 nmol/L, n−7).

Role of Sphk1 and S1P for the Myogenic Response

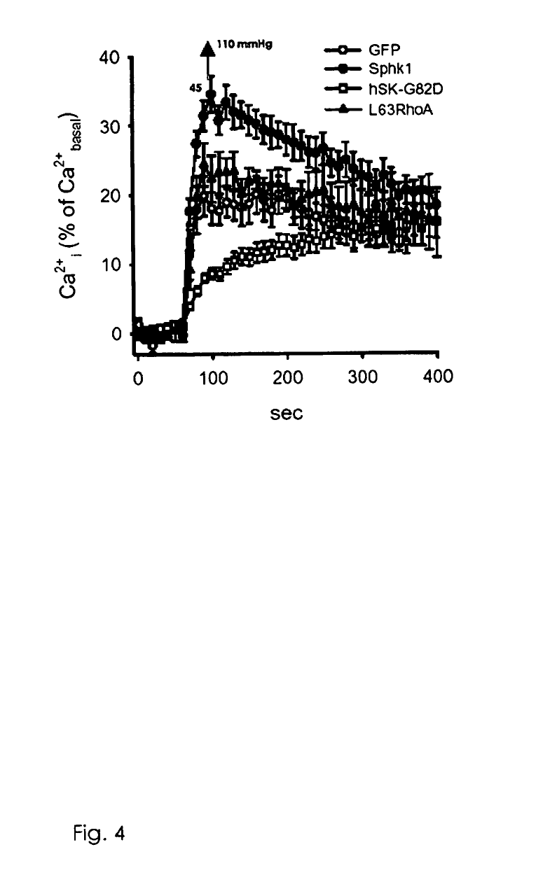

10 nmol/L SP1 induced no significant constrictions but significantly augmented myogenic responses in resistance arteries (144±11% RID, n=5). This effect was blocked by the Rho kinase inhibitor Y27632 (3±1% RID, n=4) and absent in arteries transfected with the dominant negative RhoA mutant N19RhoA (2±5% further distension, n=3), suggesting that RhoA/Rho kinase activation mediated the modulation of the myogenic response by exogenous S1P. This modulating effect was not confined to exogenous S1P since forced expression of endogenous S1P-generating Sphk1 also significantly increased myogenic responses (154±14% RID, n=12, vs. 59±9% for GFP, FIGS. 2b and 3). In contrast, myogenic responses were only residual in arteries overexpressing the dominant negative Sphk1 mutant hSK-G82D (15±5% RID, n=16 P<0.005%). Augmented myogenic responses in Sphk1-transfected arteries were associated with significantly higher initial pressure-induced increases in Ca²⁺ (maximal at 34±3% after 40 sec, n=12, FIG. 4) compared to GFP-transfected controls (20±3% after 40 sec, n=8, P<0.005%, FIG. 4). Genetic inhibition of Sphk1 with hSK-G82D significantly reduced initial increases in Ca²⁺ (9±1% after 40 sec, n=16, P<0.005, FIG. 4) leaving a slow increase in intracellular Ca²⁺ that plateaued after 4 min. After 4 min, smooth muscle Ca²⁺ levels of all groups reached the same plateau level (19±2% in Sphk1, 17±2% in GFP and 17±3% in hSK-G82D, all normalised to resting Ca²⁺ levels, FIG. 4).

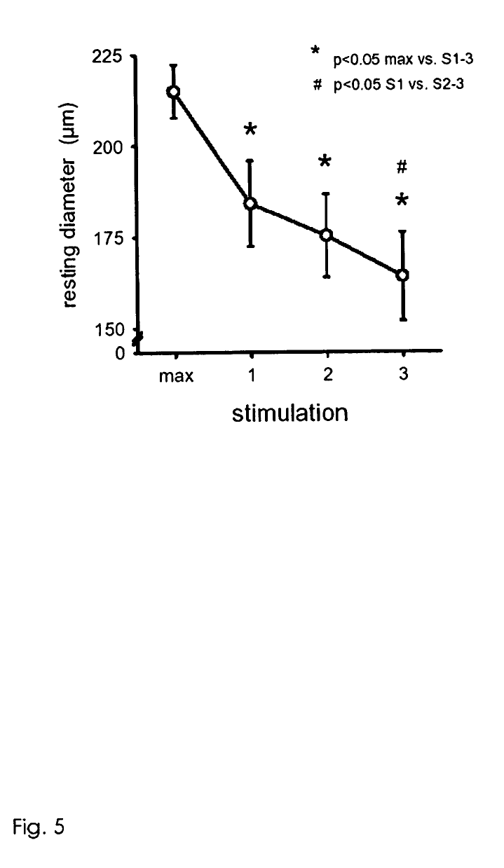

Stimulation of Sphk1-overexpressing arteries by repetitive increases in transmural pressure from 45 to 110 mmHg over 5 min intercepted by 20 min breaks progrediently increased resting tone (n=6, FIG. 5) and accelerated and strengthened myogenic responses.

Effects of Sphk1 on the Myogenic Response are Mediated by RhoA/Rho Kinase

To verify a possible contribution of RhoA or Rho kinase to the effects of Sphk1 on myogenic responses, Sphk1 was coexpressed with N19RhoA or KD1A. Both significantly inhibited the myogenic vasoconstriction (Sphk1+N19RhoA: 1±14% RID; Sphk1+KD1A: 9±4% RID, each n=8, FIG. 3).

Activation of the RhoA pathway alone, as achieved by transfection of the dominant active RhoA mutant L63Rhoa, resulted in myogenic responses of 92±12% RID, (n=8, FIG. 3) that, although greater than GFP-transfected (59±9% RID, P<0.005), were significantly smaller than in Sphk1-transfected (154±14% RID, P<0.005) arteries.

Effects of the Genetic Manipulations on Microvascular Contractility

To determine whether constrictions in transfected arteries were generally affected, constrictor responses to a single dose of 0.3 μmol/L NE were assessed in all genetically altered resistance arteries. The resulting NE-induced tone was similar in groups (GFP: 51±3%, Sphk1: 51±2%, L63RhoA: 53±1%, Sphk1+KD1A: 51±2%, Sphk1+N19RhoA: 38±10% OMD) with the exception of those arteries in which Sphk1 was genetically inhibited (hSK-G82D). They showed a marked reduction of NE-induced tone (27±6% OMD, n=16, P<0.05).

### EXAMPLE 2

### The NO-Induced Decrease in Calcium Sensitivity of Resistance Arteries is Due to Activation of the Myosin Light Chain Phosphatase and Antagonized by the RhoA/Rho Kinase Pathway

Materials and Methods

Drugs

MOPS-buffered salt solution contained (mmol/L): 145 NaCl, 4.7 KCl, 1.5 CaCl₂, 1.17 MgSO₄, 1.2 NaH₂PO₄, 2.0 pyruvate, 0.02 EDTA, 3.0 MOPS and 5.0 glucose. In “depolarizing solution” with 120 mmol/L KCl, NaCl was compensatorily reduced to 29.7 mmol/L. Fura 2-AM was purchased from Molecular Probes (Oregon, USA), Norepinephrine (NE), acetylcholine (ACh), NS1619 and sodium nitroprusside (SNP) from Sigma Chemicals (Deisenhofen, Germany). Y27632 was from Welfide Corporation, Osaka, Japan. C3 transferase and N19RhoA plasmids were kindly provided by Dr. Alan Hall, University College London, UK.

Effectene® was from Qiagen, Germany, Trans LT from Mobitec, Germany.

Concentrations given in the text refer to final bath concentrations.

Preparation of Small RA and Ca2+i and Diameter Measurements

The care of the animals and the experimental procedures were in accordance with German animal protection laws. The preparation of the vessels and the technique of calcium (fura 2) and diameter measurements were previously described (Bolz et al., 1999, supra; Bolz S S, Fisslthaler B, Pieperhoff S et al., FASEB J. 2000;14:255-260). Briefly, RA (maximal outer diameter 180-250 μm) from gracilis muscle of female hamsters were cannulated with micropipettes and studied at 45 mmHg transmural pressure. Fura 2 was alternatingly excited at 340 or 380 nm. The ratio F₃₄₀ₙₘ/F₃₈₀ₙₘ at 510 nm was calculated after subtraction of the background fluorescence (obtained after fura 2-quenching with 8 mmol/L MnCl₂). Diameters were simultaneously recorded by videomicroscopy at wavelengths>610 nm to avoid interference with fura 2-measurements.

Transfection of Intact RA

To transfect plasmids containing C3 transferase or the respective mutated RhoA sequences (N19RhoA, RhoA^{Ala-188}) into VSMCs, arteries were incubated for 18-21 h in a artery culture system (Bolz et al., 2000, supra) with culture medium containing antibiotics, the transfectant Effectene® (16 μl/ml) and 5 μg of the respective plasmid. Unspecific effects of the transfection procedure were assessed by comparing vascular responses of non-transfected RA and arteries transfected with green fluorescent protein (GFP). In arteries transfected with RhoA-GFP fusion protein all VSMCs per microscopic field showed GFP-related fluorescence (confocal microscopy, excitation 488 nm, emission 525-565 nm; FIG. 5 b).

The technique to transfect intact C3 transferase protein using Trans LT was previously described (Bolz et al., 2000, supra).

Immunofluorescence and Digital Imaging

Arteries were fixed with 3.7% formaldehyde, permeabilized with 0.3% Triton X-100, blocked with 1% BSA and incubated with the primary antibody (MLCP: rabbit anti-mouse, 1:200, Covance; RhoA: mouse monoclonal, 1:200, Santa Cruz Biotechnology). FITC-labelled goat anti-rabbit or donkey anti-mouse (1:200, each) were used as secondary antibodies. Images were obtained using a Zeiss LSM410 confocal microscope equipped with a Kr/Ar laser and a 40×/1.2 W water immersion objective.

Immunoblotting

Tissue samples of hamster aorta were quick-frozen in liquid nitrogen and homogenized. Cytosolic and particulate fractions were separated by centrifugation of the homogenate at 100.000 g (Beckman Coulter, Optima Max-E). Pellets were resuspended in lysis buffer plus 1% Triton-X 100. Protein-matched samples were electrophoresed by SDS-PAGE (7%), transferred to nitrocellulose membranes (Amersham), and subjected to immunostaining using a polyclonal primary antibody (rabbit anti-mouse, 1:500). An HRP-labelled secondary antibody (goat anti-rabbit, 1:10000, Santa Cruz) was used with ECLplus (Amersham) to visualize the signal.

Experimental Protocols

Changes in diameter and [Ca²⁺]ᵢ were continuously recorded in 70 vessels from 41 animals. All vessels studied developed spontaneous tone (9.6±1% of maximal diameter). The viability of each vessel was assessed by its constriction to NE (0.3 μmol/L) and a dilation>80% in response to 1 μmol/L ACh.

The apparent Ca²⁺-sensitivity of the arteries was assessed by stepwise increasing the extracellular Ca²⁺ concentration (Ca^{2+, 0}-3 mmol/L) around the arteries kept in depolarizing solution (120 mmol/L K⁺). Depolarization-dependent opening of voltage-gated calcium channels allowed increases in Ca²⁺ ex to be reproducibly followed by increases in VSMC [Ca²⁺]ᵢ (Bolz et al., 2000, supra). The Ca²⁺-sensitivity was assessed under control conditions, in the presence of SNP and in the combined presence of SNP and the respective modulating substance or protein (ODQ, calyculin A, S1P, RhoA^{Ala-188}).

Additionally, dose-response curves for SNP were obtained in arteries preconstricted by 0.3 μmmol/L NE under control conditions, in the presence of the Rho kinase inhibitor Y27632 (1 μmol/L) or in N19RhoA-transfected arteries.

Statistical Analysis

Dilations are expressed as “% of maximum dilation”=[(dia_{VD}−dia_{NE})/(diaₘₐₓ−dia_{NE})]×100, with dia_{VD} and dia_{NE} representing steady state diameters 2 min after administration of NE or the respective vasodilator and diaₘₐₓ being the maximal diameter obtained in Ca²⁺-free 1 mmol/L EGTA-containing MOPS buffer.

Due to methodological uncertainties in calculating exact values for [Ca²⁺]ᵢ in intact vessels (Meininger G A, Zawieja D C, Falcone J C et al., Am J. Physiol. 1991; 261:H950-H959), fluorescence ratios (F₃₄₀ₙₘ/F₃₈₀ₙₘ) are presented instead. Calibration curves obtained in a cell free system indicated that the range of ratios observed here (0.4-6.3) fitted into the linear range of the curve that comprises physiological intracellular Ca²⁺ concentrations (42.2 to 1520 nmol/L).

Steady state values from different groups were compared with ANOVA followed by post hoc analysis of the means. Data are presented as mean±SEM. Differences were considered significant at an error probability of p<0.05.

Curves were compared using a non-linear regression analysis applied first to every individual curve and then to the pooled data. Curves were considered to be different if the F-test indicated a significantly smaller sum of squares for the deviations in each individual fit as compared to the deviation in the fit to the pooled data (Motulsky et al., 1987, supra).

Results

The NO-Induced Desensitization of the Contractile Apparatus is Dependent on cGMP

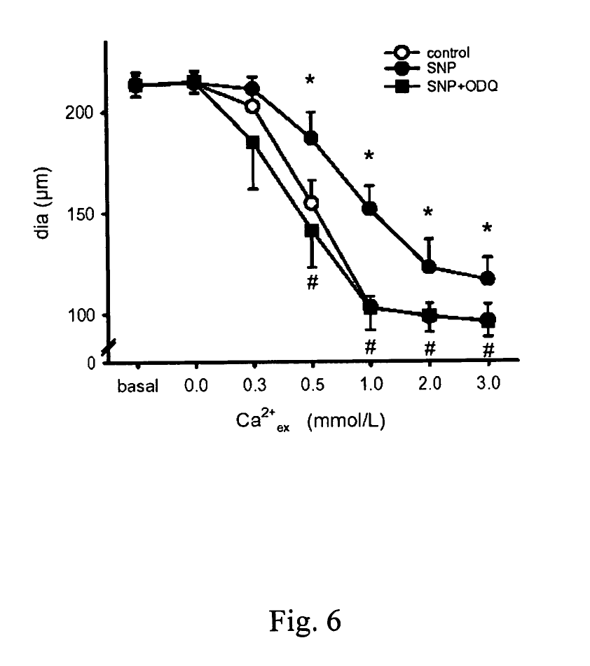

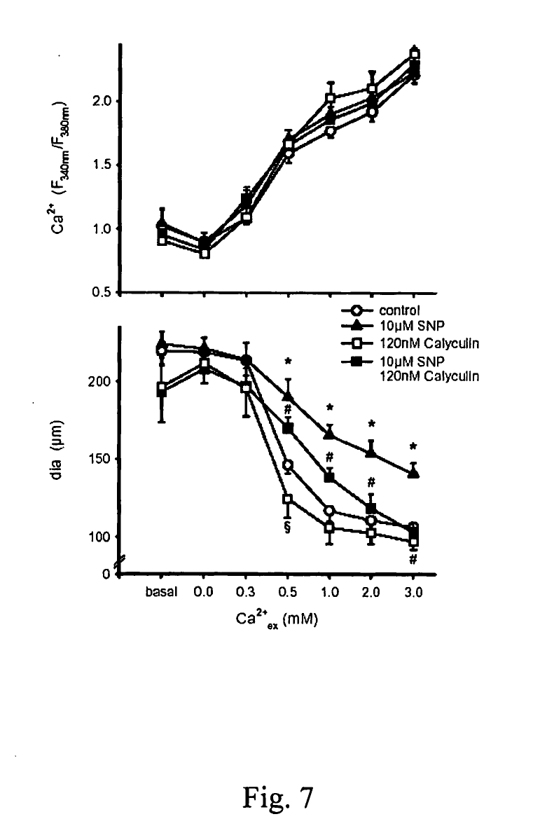

Stepwise constrictions of K⁺-depolarized arteries occurring in parallel to increases in Ca²⁺ₑₓ were significantly attenuated in the presence of 10 μmol/L SNP (p<0.05, n=7, FIG. 6). Increases in [Ca²⁺]ᵢ were virtually identical in control and SNP-treated arteries for any given concentration of Ca²⁺ₑₓ (FIG. 7), suggesting that NO decreased the myofilament Ca²⁺-sensitivity. This NO effect was entirely mediated by cGMP because it was blocked following inhibition of the soluble guanylate cyclase by ODQ (1 μmol/L, p<0.05, n=7, FIG. 6). [Ca²⁺]ᵢ was not significantly different in control, SNP- or SNP/ODQ-treated RA.

MLCP Mediates the Ca2+-Desensitizing Effect of NO

The potential involvement of the MLCP in NO-induced Ca²⁺-desensitization was assessed in RA pretreated with the MLCP inhibitor calyculin A at a concentration (120 nmol/L) considered to be specific for the MLCP (Ishihara H, Martin B L, Brautigan D L et al., Biochem Biophys Res Commun. 1989; 159:871-877). Calyculin A almost abolished the desensitizing effect of NO (p<0.05, n=7, FIG. 7), suggesting that this effect requires a fully functional MLCP. None of the myofilament Ca²⁺-sensitivity-modulating treatments affected VSMC [Ca²⁺]ᵢ (FIG. 2).

Activation of RhoA/Rho Kinase Antagonizes NO-Induced Desensitization and Dilations

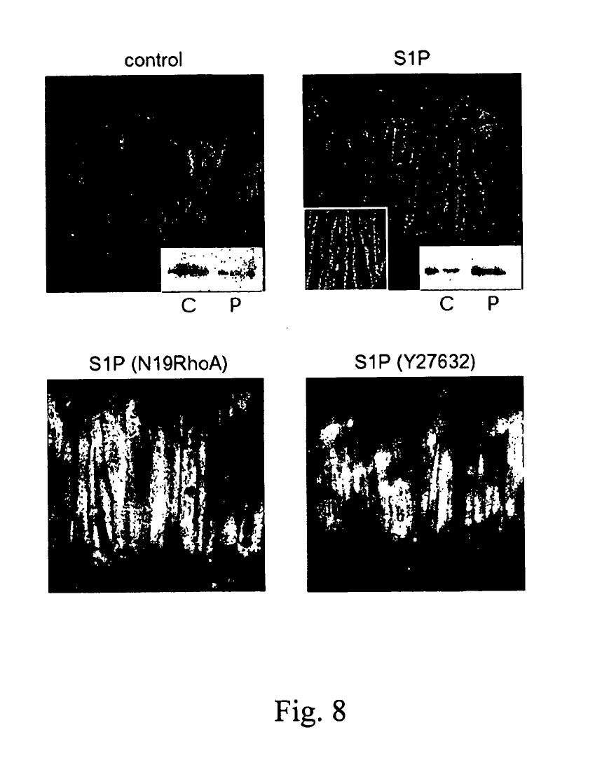

At concentrations<1 μmol/L the sphingolipid mediator S1P induced constrictions of RA that were abolished after treatment with the RhoA inhibitor C3 transferase (n=7) or the Rho kinase inhibitor Y27632 (n=7, Table 2). S1P-induced activation of RhoA/Rho kinase induced a translocation of the MLCP subunit MYPT1 to the VSMC plasmamembrane (FIG. 8), an effect that has recently been linked to inhibition of MLCP (Shin H M, Je H D, Gallant C et al., Circ Res. 2002; 90:546-553). S1P-induced translocation was absent in arteries transfected with the dominant-negative RhoA mutant N19RhoA and those pretreated with Y27632 (1 μmol/L, FIG. 8).

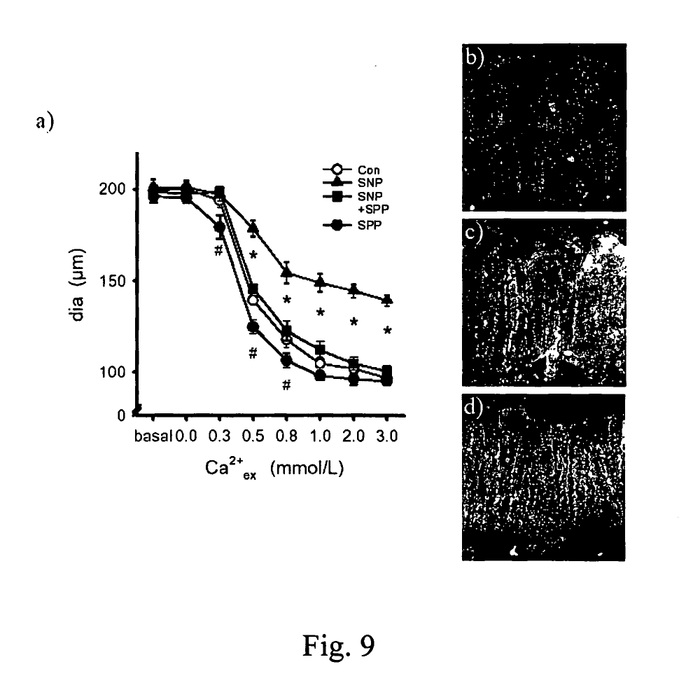

S1P (10 nmol/L, n=11) which per se increased the Ca²⁺-sensitivity only in a medium concentration range of Ca²⁺ₑₓ (0.25-0.75 mmol/L) abolished the NO-induced Ca²⁺-desensitization over the whole range of Ca²⁺ ex (FIG. 9a).

RhoA showing a cytosolic localisation under resting conditions was translocated to the membrane following stimulation with 10 nmol/L S1P (FIG. 9b,c). This translocation was not affected by subsequent addition of SNP (10 μmol/L, 3 min, FIG. 9d).

SNP(1 μmol/L)-induced dilations after preconstriction with 1 μmol/L S1P were significantly smaller (by 64±8%, n=4) than those after preconstriction with 0.3 μmol/L NE despite virtually identical preconstriction levels (130±10 vs. 128±2 μm, n=4).

Transfection of RhoAAla-188 Does Not Affect NO-Induced Ca2+-Desensitizing Effects

Recently, Sauzeau et al. showed that in permeabilized aortic rings SNP directly inactivated RhoA through a cGK 1-mediated translocation of activated RhoA back to the cytosol (Sauzeau V, Le J H, Cario-Toumaniantz C et al. Cyclic GMP-dependent Protein Kinase Signalling Pathway Inhibits RhoA-induced Ca2+ Sensitization of Contraction in Vascular Smooth Muscle. J Biol. Chem. 2000;275:21722-21729). To test the involvement of this mechanism in desensitizing effects of NO in RA, intact RA were transfected with RhoA^{Ala-188} (n=6) that cannot be phosphorylated by cGK I. Desensitizing effects of NO were fully maintained in RhoA^{Ala-188}-transfected RA (FIG. 9e). VSMC [Ca^{2+]}ᵢ was not affected by SNP.

Inactivation of RhoA/Rho Kinase Augments Dilations Induced by NO

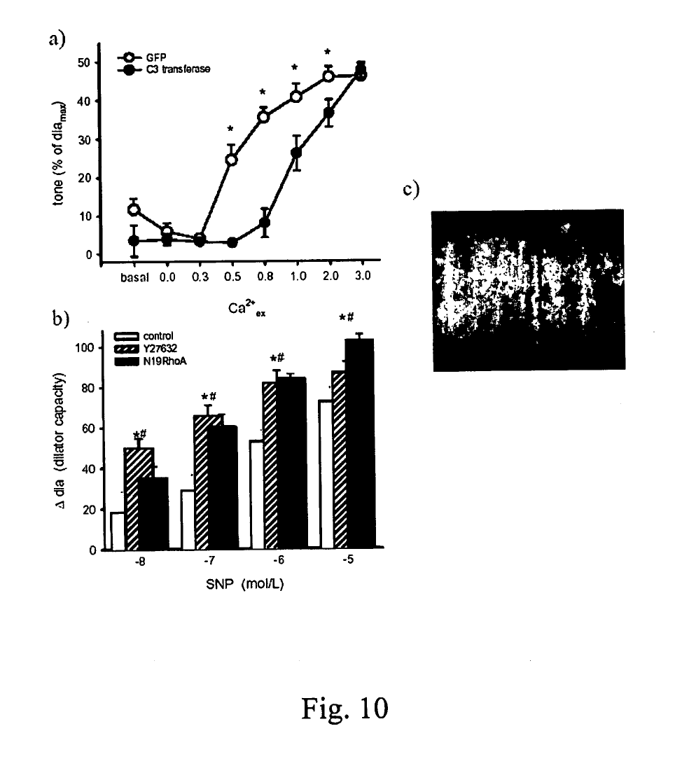

The Ca²⁺/diameter curve in C3 transferase-transfected RA was significantly shifted to the right (FIG. 10a) suggesting a high basal activity of RhoA/Rho kinase (Gong M C, Fujihara H, Somlyo A V et al., J Biol Chem. 1997; 272:10704-10709). To test whether this high basal activity antagonized Ca²⁺-desensitizing and dilatory effects of NO under resting conditions, NO-induced dilations were studied in arteries treated with Y27632 (n=5) or transfected with N19RhoA (n=6). These inhibitions of RhoA/Rho kinase significantly augmented NO-induced dilations (FIG. 10c).

Genetic inhibition of RhoA by transfection with N19RhoA did not affect Ca²⁺-dependent dilations induced by ACh (0.01-1 μmol/L in the presence of L-NA/indomethacin, 30 μmol/L, each, n=4, FIG. 10d) or the K_{Ca}channel opener NS1619 (1-100 μmol/L, n=4, FIG. 10d). Dilations by ACh (+L-NA/indomethacin) and NS1619 were abolished in the presence of the K_{Ca}channel inhibitor charybdotoxin (n=4 each, p<0.001).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bayliss, W. M. (1919) J Physiol Lond 28:220-231.
Bolz S S, Fisslthaler B, Pieperhoff S et al. Antisense oligonucleotides against cytochrome P450 2C8 attenuate EDHF-mediated Ca(2+) changes and dilation in isolated resistance arteries. FASEB J. 2000;14:255-260.
Bolz, S. S., de Wit, C., Pohl, U. (1999) Br J Pharmacol 128:124-134.
Bolz, S. S., Galle, J., Derwand, R. et al. (2000) Circulation 102:2402-2410.
Bolz, S. S., Pieperhoff, S., de Wit, C. et al. (2000) Am J Physiol Heart Circ Physiol 279:H1434-H1439.
Bunin B A, et al. (1994) Proc. Natl. Acad. Sci. USA, 91:4708-4712
DeWitt S H, et al. (1993) Proc. Natl. Acad. Sci. USA, 90:6909-6913
Gong M C, Fujihara H, Somlyo A V et al. Translocation of RhoA associated with Ca²⁺ sensitization of smooth muscle. J Biol Chem. 1997;272:10704-10709.
Ishihara H, Martin B L, Brautigan D L et al. Calyculin A and okadaic acid: inhibitors of protein phosphatase activity. Biochem Biophys Res Commun. 1989;159:871-877.
Meininger G A, Zawieja D C, Falcone J C et al. Calcium measurement in isolated arterioles during myogenic and agonist stimulation. Am J Physiol. 1991;261:H950-H959.
Metting, P. J., Stein, P. M., Stoos, B. A. et al. (1989) Am J Physiol 256:R98-R105.
Motulsky, H. J., Ransnas, L. A. (1987) FASEB J 1:365-374.
Pitson, S. M., Moretti, P. A., Zebol, J. R. et al. (2000) J Biol Chem 275:33945-33950.
Sauzeau V, Le J H, Cario-Toumaniantz C et al. Cyclic GMP-dependent Protein Kinase Signalling Pathway Inhibits RhoA-induced Ca2+ Sensitization of Contraction in Vascular Smooth Muscle. J Biol Chem. 2000;275:21722-21729.

Shin H M, Je H D, Gallant C et al. Differential association and localization of myosin phosphatase subunits during agonist-induced signal transduction in smooth muscle. Circ Res. 2002;90:546-553.

TABLE 2
Sphingosine-1-phosphate-induced vasoconstrictions < 1
μmol/L were reduced after inhibition of RhoA (C3 transferase,
n = 7) or Rho kinase (Y27632, n = 7).

| S1P | Control | Y27632 | C3 transferase |
|---|---|---|---|
| μmol/L | % of diaₘₐₓ | % of diaₘₐₓ | % of diaₘₐₓ |
| 0.001 | −0.2 ± 0.4 | 0.1 ± 0.4 | 0.7 ± 0.3 |
| 0.01 | −7.7 ± 2.4 | −0.4 ± 0.2 * | 0.5 ± 0.6 * |
| 0.1 | −27.0 ± 3.4 | −3.4 ± 1.8 * | −2.8 ± 0.7 * |
| 1 | −45.0 ± 2.0 | −20.0 ± 7.2 | −18.1 ± 2.9 * |

(mean ± SEM, significant differences between groups (p < 0.05) are indicated by * for C3 and Y27632 vs. control)

1. A method of modulating mammalian smooth muscle cell activity, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

2. A method of regulating smooth muscle cell activity in a mammal, said method comprising modulating the functional activity of sphingosine kinase mediated signalling in said mammal wherein upregulating sphingosine kinase mediated signalling activity to a functionally effective level upregulates said smooth muscle cell activity and downregulating sphingosine kinase mediated signalling to a functionally ineffective level downregulates said smooth muscle cell activity.

3. A method for the treatment and/or prophylaxis of a condition characterised by aberrant, unwanted or otherwise inappropriate smooth muscle cell activity in a mammal, said method comprising modulating the functional activity of sphingosine kinase mediated signalling wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

4. The method according to claim 1 or 2 or 3 wherein said smooth muscle cell is a vascular, bronchial or intestinal smooth muscle cell.

5. The method according to claim 4 wherein said vascular smooth muscle cell is an arterial smooth muscle cell.

6. The method according to claim 1 or 2 or 3 wherein said smooth muscle cell is a gastric, bladder or uterine smooth muscle cell.

7. The method according to any one of claims 1 to 6 wherein said smooth muscle cell activity is smooth muscle cell tone.

8. The method according to claim 7 wherein said smooth muscle cell is a bronchial smooth muscle cell and said modulation of bronchial smooth muscle cell tone is downregulation of tone.

9. The method according to claim 7 wherein said smooth muscle cell is a vascular smooth muscle cell and said modulation of vascular smooth muscle cell tone is downregulation of tone.

10. The method according to claim 7 wherein said smooth muscle cell is a vascular smooth muscle cell and said modulation of vascular smooth muscle cell tone is upregulation of tone.

11. The method according to claim 3 wherein said condition is excessive arterial resistance, said smooth muscle cell activity is arterial smooth muscle cell tone and said modulation of sphingosine kinase activity is downregulation of activity.

12. The method according to claim 11 wherein said condition is high blood pressure.

13. The method according to claim 12 wherein said high blood pressure correlates to hypertension.

14. The method according to claim 3 wherein said condition is inadequate arterial resistance, said smooth muscle cell activity is arterial smooth muscle cell tone and said modulation of sphingosine kinase activity is upregulation of activity.

15. The method according to claim 14 wherein said condition is hypotension or septic shock.

16. The method according to claim 15 wherein said hypotension is related to Waterhouse-Friedrichsen syndrome.

17. The method according to claim 15 wherein said septic shock is related to bacterial meningitis.

18. The method according to claim 3 wherein said condition is excessive bronchial constriction, said smooth muscle cell activity is bronchial smooth muscle cell tone and said modulation of sphingosine kinase activity is downregulation.

19. The method according to claim 18 wherein said excessive bronchial constriction is related to asthma, allergy or anaphylactic shock.

20. The method according to any one of claims 1-7, 10 or 14-17 wherein said modulation is upregulation of sphingosine kinase mediated signalling and said upregulation is achieved by introducing into said smooth muscle cell a nucleic acid molecule encoding sphingosine kinase or functional equivalent, derivative or homologue thereof or the sphingosine kinase expression product or functional derivative, homologue, analogue, equivalent or mimetic thereof.

21. The method according to any one of claims 1-19 wherein said modulation is achieved by contacting said smooth muscle cell with a proteinaceous or non-proteinaceous molecule which modulates transcriptional and/or translational regulation of the sphingosine kinase gene.

22. The method according to any one of claims 1-7, 10 or 14-17 wherein said modulation is upregulation of sphingosine kinase levels and said upregulation is achieved by contacting said smooth muscle cell with a proteinaceous or non-proteinaceous molecule which functions as an agonist of the sphingosine kinase expression product.

23. The method according to any one of claims 1-9, 11-13 or 18-19 wherein said modulation is downregulation of sphingosine kinase mediated signalling and said downregulation is achieved by contacting said smooth muscle cell with a proteinaceous or non-proteinaceous molecule which functions as an antagonist to the sphingosine kinase expression product.

24. The method according to claim 23 wherein said molecule is a mutant sphingosine kinase which mutant is characterised by substitution of the glycine residue at position 82 to aspartate.

25. The method according to claim 1 wherein said smooth muscle cell activity is modulated in vivo.

26. The method according to claim 1 wherein said smooth muscle cell activity is modulated in vitro.

27. Use of an agent capable of modulating the functionally effective level of sphingosine kinase mediated signalling in the manufacture of a medicament for the regulation of vascular smooth muscle cell activity in a mammal wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

28. Use of an agent capable of modulating the functionally effective level of sphingosine kinase mediated signalling in the manufacture of a medicament for the treatment of a condition characterised by aberrant, unwanted or otherwise inappropriate smooth muscle cell activity in a mammal wherein upregulating sphingosine kinase mediated signalling to a functionally effective level upregulates said smooth muscle cell activity and downregulating sphingosine kinase mediated signalling to a functionally ineffective level downregulates said smooth muscle cell activity.

29. Use according to claim 27 or 28 wherein said smooth muscle cell is a vascular, bronchial or intestinal smooth muscle cell.

30. Use according to claim 29 wherein said vascular smooth muscle cell is an arterial smooth muscle cell.

31. Use according to claim 27 or 28 wherein said smooth muscle cell is a gastric, bladder or uterine smooth muscle cell.

32. Use according to any one of claims 27-31 wherein said smooth muscle cell activity is smooth muscle cell tone.

33. Use according to claim 32, wherein said smooth muscle cell is a bronchial smooth muscle cell and said modulation of bronchial smooth muscle cell tone is downregulation of tone.

34. Use according to claim 32 wherein said smooth muscle cell is a vascular smooth muscle cell and said modulation of vascular smooth muscle cell tone is downregulation of tone.

35. Use according to claim 32 wherein said smooth muscle cell is a vascular smooth muscle cell and said modulation of vascular smooth muscle cell tone is upregulation of tone.

36. Use according to claim 28 wherein said condition is excessive arterial resistance, said smooth muscle cell activity is arterial smooth muscle cell tone and said modulation of sphingosine kinase activity is downregulation of activity.

37. Use according to claim 36 wherein said condition is high blood pressure.

38. Use according to claim 37 wherein said high blood pressure correlates to hypertension.

39. Use according to claim 28 wherein said condition is inadequate arterial resistance, said smooth muscle cell activity is arterial smooth muscle cell tone and said modulation of sphingosine kinase activity is upregulation of activity.

40. Use according to claim 39 wherein said condition is hypotension or septic shock.

41. Use according to claim 40 wherein said hypotension is related to Waterhouse-Friedrichsen syndrome.

42. Use according to claim 40 wherein said septic shock is related to bacterial meningitis.

43. Use according to claim 28 wherein said condition is excessive bronchial constriction, said smooth muscle cell activity is bronchial smooth muscle cell tone and said modulation of sphingosine kinase activity is downregulation.

44. Use according to claim 43 wherein said excessive bronchial constriction relates to asthma, allergy or anaphylactic shock.

45. Use according to any one of claims 27-32, 35 or 39-42 wherein said modulation is upregulation of sphingosine kinase mediated signalling and said upregulation is achieved by introducing into said smooth muscle cell a nucleic acid molecule encoding sphingosine kinase or functional equivalent, derivative or homologue thereof or the sphingosine kinase expression product or functional derivative, homologue, analogue, equivalent or mimetic thereof.

46. Use according to any one of claims 27-44 wherein said modulation is achieved by contacting said smooth muscle cell with a proteinaceous or non-proteinaceous molecule which modulates transcriptional and/or translational regulation of the sphingosine kinase gene.

47. Use according to any one of claims 27-32, 35 or 39-42 wherein said modulation is upregulation of sphingosine kinase levels and said upregulation is achieved by contacting said smooth muscle cell with a proteinaceous or non-proteinaceous molecule which functions as an agonist of the sphingosine kinase expression product.

48. Use according to any one of claims 27-34, 36-38 or 43-44 wherein said modulation is downregulation of sphingosine kinase mediated signalling and said downregulation is achieved by contacting said smooth muscle cell with a proteinaceous or non-proteinaceous molecule which functions as an antagonist to the sphingosine kinase expression product.

49. Use according to claim 48 wherein said molecule is a mutant sphingosine kinase which mutant is characterised by substitution of the glycine residue at position 82 to aspartate.

50. Use according to claim 27 wherein said smooth muscle cell activity is modulated in vivo.

51. Use according to claim 27 wherein said smooth muscle cell activity is modulated in vitro.

52. A pharmaceutical composition comprising the modulatory agent as hereinbefore defined and one or more pharmaceutically acceptable carriers and/or diluents.
